(19) United States
(12) Patent Application Publication
(10) Pub. No.: US 2022/0148722 A1
(43) Pub. Date: May 12, 2022

(54) CARE ASSISTANCE DEVICE, CARE ASSISTANCE METHOD, AND CARE ASSISTANCE SYSTEM

(71) Applicant: Nippon Telegraph and Telephone Corporation, Tokyo (JP)

(72) Inventors: Takayuki Ogasawara, Tokyo (JP); Junichi Kodate, Tokyo (JP)

(21) Appl. No.: 17/433,838

(22) PCT Filed: Feb. 12, 2020
(86) PCT No.: PCT/JP2020/005287

§ 371 Date: Aug. 25, 2021

(30) Foreign Application Priority Data
Feb. 27, 2019 (JP) 2019-033933

(51) Int. Cl. G16H 40/63; A61B 5/00; A61B 5/11

(52) U.S. Cl. CPC G16H 40/63; A61B 5/74; A61B 5/1116; A61B 5/4809; A61B 5/4812

(57) ABSTRACT

A long-term care support device includes: a sensor data acquisition device that acquires biological information of a user that was measured by a sensor; a sleep stage detection device that detects a sleep stage of the user based on the acquired biological information; a timing determination device that determines that it is time to change the position of the user if the sleep stage of the user is the deep sleep stage; and a notification device that, if the timing determination device determined that it is time to change the position of the user, gives a notification to such effect

### CROSS-REFERENCE TO RELATED APPLICATIONS

This application is a national phase entry of PCT Application No. PCT/JP2020/005287, filed on Feb. 12, 2020, which claims priority to Japanese Application No. 2019-033933, filed on Feb. 27, 2019, which applications are hereby incorporated herein by reference.

### TECHNICAL FIELD

The present invention relates to a long-term care support device, a long-term care support method, and a long-term care support system.

### BACKGROUND

In long-term care, it is important to prevent the formation of bedsores in users such as bedridden patients who have difficulty turning over on their own. Conventionally, in order to prevent bedsores, it is known to be necessary to change the user's position at regular intervals such as every two to four hours (see NPL 1). For example, a caregiver regularly visits a bedridden user and manually changes the position of the user. There are also cases where a roll-over support bed that uses a motor to change the user's position is used (see NPL 9).

### CITATION LIST

### Non Patent Literature

NPL 1 “Bedsore Prevention: Differences Between Position Change Every 2 Hours and Position Change Every 4 Hours”, [online], [retrieved Dec. 15, 2018], Internet <https://kango-oshigoto.jp/hatenurse/article/1207/>.

NPL 2 Arinobu NIIJIMA, et al., “A Study of Sleep-Wake Detection Algorithm Based on Activity Counts and Idleness Periods with an Accelerometer”, IEICE Technical Report, Vol. 115, No. 486, LOIS 2015-63, pp. 1-6, March 2016.

NPL 3 Toki TAKEDA, et al., “Time-dependent Sleep Stage Transition Model Based on Heart Rate Variability”, DEIM Forum 2015 G6-5, [online], [retrieved Feb. 15, 2019], Internet <http://db-event.jpn.org/deim2015/paper/24.pdf>.

NPL 4 Kazuki SUZUMURA, “Sleep Stage Estimation by Heart Rate Variability (HRV) Using Microwave Radar: Sleep Stage Identification By Pulse Extraction and SVM Utilization Through Variable Moving Average Difference Method”, Tokyo Metropolitan University Graduate School, 2016 Thesis.

NPL 5 Shima OKADA, et al., “Noncontact Sleep Stage Measurement Using Microwave Sensing”, Japanese Society for Medical and Biological Engineering, 2016, Vol. 54(3), pp. 139-144, published November 23, 2016, Online ISSN 1881-4379, Print ISSN 1347-443X

NPL 6 Takashi WATANABE, et al., “Estimation of the Sleep Stages by the Non-Restrictive Air Mattress Sensor”, The Society of Instrument and Control Engineers, 2001, Vol. 37(9), pp. 821-828, published Mar. 27, 2009, Online ISSN 1883-8189, Print ISSN 0453-4654.

NPL 7 Hisayoshi SUGAWARA, et al., “Impact of nurse-assisted patient turning at different sleep stages on the quality of subsequent sleep”, Conf. Proc. IEEE Eng Med Biol Soc., July 2018, 2018: 2096-2099, doi: 10.1109/EMBC.2018.8512679.

NPL 8 Japan Medical Association, “Science of Sleep”, [online], [retrieved Feb. 15, 2019], Internet <https://www.med.or.jp/forest/health/live/05.html>.

NPL 9 “Automatic Roll-over Support Bed”, [online], [retrieved Nov. 30, 2018], Internet <https://kaigorobot-online.com/contents/123>.

### SUMMARY

### Technical Problem

However, in conventional technology, changing the position of a sleeping user sometimes causes the user to wake up, and once awakened, the user may have difficulty in falling asleep again, which leads to the problem of a large burden on the user.

Embodiments of the present invention were achieved in order to solve the foregoing problems, and an object of the present invention is to provide long-term care support technology that can change the position of a sleeping user while also alleviating the burden on the user.

### Means for Solving the Problem

In order to solve the foregoing problems, a long-term care support device according to an aspect of embodiments of the present invention includes: a sensor data acquisition unit configured to acquire biological information of a user that was measured by a sensor; a sleep stage detection unit configured to detect a sleep stage of the user based on the acquired biological information; a timing determination unit configured to determine that it is time to change a position of the user if the sleep stage of the user is a deep sleep stage; and a notification unit configured to, if the timing determination unit determined that it is time to change the position of the user, give a notification to such effect.

Also, in the long-term care support device according to an aspect of embodiments of the present invention, the sleep stage detection unit may be further configured to detect that the user is in a sleep onset stage, the long-term care support device may further include a first clock unit configured to measure a preset first period from a time when it was detected that the user is in the sleep onset stage, and if the time measured by the first clock unit has reached the first period, the timing determination unit may determine that it is time to change the position of the user.

Also, the long-term care support device according to an aspect of embodiments of the present invention may further include: a change determination unit configured to determine that the position of the user changed based on the sleep stage of the user that was detected by the sleep stage detection unit; and a second clock unit configured to measure a preset second period from a time when the change determination unit detected that the position of the user changed, wherein if the sleep stage of the user is a deep sleep stage, or if the time measured by the second clock unit has reached the second period, the timing determination unit may determine that it is time to change the position of the user.

Also, in the long-term care support device according to an aspect of embodiments of the present invention, if the sleep stage detection unit detects that the user is in the sleep onset stage and thereafter detects that the user is in an awake stage, the timing determination unit may determine that it is time to change the position of the user.

Also, the long-term care support device according to an aspect of embodiments of the present invention may further include: a position change processing unit configured to output a control signal to a movable device configured to change the position of the user, based on a notification that it is time to change the position of the user.

Also, in the long-term care support device according to an aspect of embodiments of the present invention, the biological information acquired by the sensor data acquisition unit may be the biological information measured by the sensor that is at least any of a heart rate monitor, a 3-axis acceleration sensor, a microwave sensor, and a pressure sensor.

In order to solve the foregoing problems, a long-term care support method according to an aspect of embodiments of the present invention includes: a first step of acquiring biological information of a user that was measured by a sensor; a second step of detecting a sleep stage of the user according to a preset reference based on the biological information acquired in the first step; a third step of determining that it is time to change a position of the user if the sleep stage of the user that was detected in the second step is a deep sleep stage; and a fourth step of, if it was determined in the third step that it is time to change the position of the user, giving a notification to such effect.

In order to solve the foregoing problems, a long-term care support system according to an aspect of embodiments of the present invention includes: a sensor terminal configured to output, to an external device, biological information of a user that was measured by a sensor; a relay terminal configured to receive the biological information output by the sensor terminal, and output the biological information to an external device; and an external terminal configured to receive the biological information that was output by the sensor terminal or the relay terminal, and display the biological information on a display device, wherein at least any one of the sensor terminal, the relay terminal, and the external terminal includes: a sensor data acquisition unit configured to acquire the biological information, a sleep stage detection unit configured to detect a sleep stage of the user according to a preset reference based on the acquired biological information, a timing determination unit configured to determine that it is time to change the position of the user if the sleep stage of the user that was detected is a deep sleep state, and a notification unit configured to, if the timing determination unit determined that it is time to change the position of the user, give a notification to such effect.

Effects of embodiments of the Invention

According to embodiments of the present invention, if the sleep stage of the user detected based on the biological information of the user is the deep sleep stage, it is determined that it is time to change the body position of the user, and a notification is given, thus making it possible to change the posture of the sleeping user while also alleviating the burden on the user.

### BRIEF DESCRIPTION OF THE DRAWINGS

FIG. 1 is a block diagram showing a functional configuration of a long-term care support device according to a first embodiment of the present invention.

FIG. 2 is a block diagram showing a configuration of a data analysis unit according to the first embodiment.

FIG. 3 is a block diagram showing an example of a configuration of a computer that realizes the long-term care support device according to the first embodiment.

FIG. 4 is a flowchart illustrating operations of the long-term care support device according to the first embodiment of the present invention.

FIG. 5 is a diagram for illustrating an overview of a configuration of a long-term care support system according to the first embodiment.

FIG. 6 is a block diagram showing a configuration of the long-term care support system according to the first embodiment.

FIG. 7 is a sequence diagram for illustrating operations of the long-term care support system according to the first embodiment.

FIG. 8 is a block diagram showing a functional configuration of a long-term care support device according to a second embodiment of the present invention.

FIG. 9 is a flowchart illustrating operations of the long-term care support device according to the second embodiment of the present invention.

FIG. 10 is a block diagram showing a configuration of the long-term care support system according to the second embodiment.

FIG. 11 is a block diagram showing a configuration of a data analysis unit according to a third embodiment.

FIG. 12 is a flowchart illustrating operations of the long-term care support device according to the third embodiment of the present invention.

FIG. 13 is a flowchart illustrating operations of the long-term care support device according to a fourth embodiment of the present invention.

FIG. 14 is a block diagram showing a functional configuration of a long-term care support device according to a fifth embodiment of the present invention.

FIG. 15 is a diagram for illustrating an overview of a configuration of a long-term care support system according to the fifth embodiment.

FIG. 16 is a block diagram showing a configuration of the long-term care support system according to the fifth embodiment.

FIG. 17 is a sequence diagram for illustrating operations of the long-term care support system according to the fifth embodiment.

### DETAILED DESCRIPTION OF ILLUSTRATIVE EMBODIMENTS

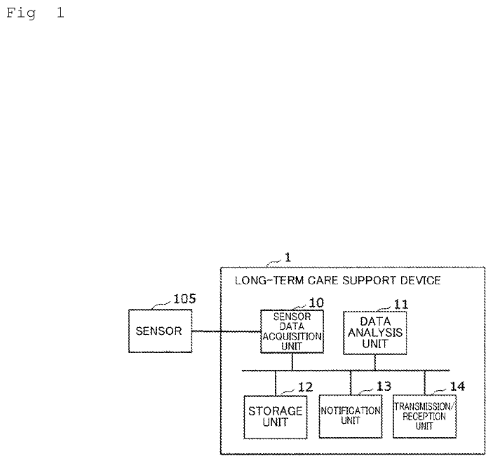

Hereinafter, preferred embodiments of the present invention will be described in detail with reference to FIGS. 1 to 17.

### First Embodiment

First, an overview of the configuration of a long-term care support device 1 according to a first embodiment of the present invention will be described. FIG. 1 is a block diagram showing a functional configuration of the long-term care support device 1. The long-term care support device 1 acquires biological information of a user, which is measured by a sensor, analyzes the user's sleep, and gives a notification that it is time for a position change if the user is in the deep sleep stage.

In the present embodiment, “user” refers to, for example, an elderly person or a patient who is in a state where it is difficult to turn over on their own. Also, by receiving a notification on a terminal or the like, a caregiver or a nurse who cares for the user can carry out the position change after finding out a more suitable timing for changing the position of the user.

### Function Blocks of Long-Term Care Support Device

The long-term care support device 1 includes a sensor data acquisition unit 10, a data analysis unit 11, a storage unit 12, a notification unit 13, and a transmission/reception unit 14.

The sensor data acquisition unit 10 acquires biological information of a user that was measured by a sensor 105. More specifically, if a heart rate monitor is attached to the user as the sensor 105, for example, the sensor data acquisition unit 10 calculates a heart rate variability from an electrocardiographic waveform based on the electrocardiographic potential measured by the heart rate monitor. Also, if a 3-axis acceleration sensor is attached to the user as the sensor 15, the sensor data acquisition unit 10 converts an analog acceleration signal measured by the acceleration sensor into a digital signal at a predetermined sampling rate. The biological information measured by the sensor data acquisition unit 10 is stored in a later-described storage unit 12 in association with a measurement time.

The sensor data acquisition unit 10 can acquire microwave data and pressure data as biological information of the user in addition to heart rate variability and triaxial acceleration.

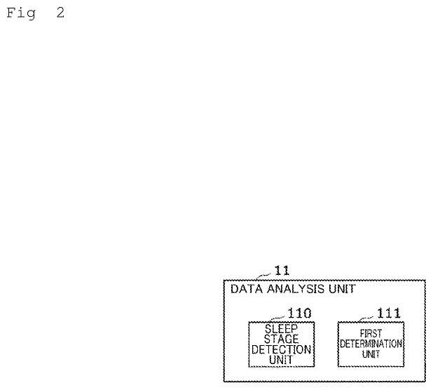

The data analysis unit 11 analyzes the biological information of the user acquired by the sensor data acquisition unit 10, detects a sleep stage that indicates the depth of sleep of the user, and determines whether or not it is time to change the position of the user. As shown in FIG. 2, the data analysis unit 11 includes a sleep stage detection unit no and a first determination unit (timing determination unit) 111.

The sleep stage detection unit no detects a sleep stage indicating the depth of sleep of the user based on the biological information acquired by the sensor data acquisition unit 10. The sleep stage detection unit no detects the sleep stage of the user based on a threshold value setting or the like that corresponds to the type of sensor data acquired by the sensor data acquisition unit 10. The sleep stage detection unit no may also be configured to sequentially update the sleep stage of the user at regular intervals.

If the sensor 105 is a heart rate monitor, the sleep stage detection unit no uses a Temporal-dependent Sleep Stage Transition Model (TSST), for example, to classify the sleep stage based on the heart rate variability acquired by the sensor data acquisition unit 10 (see NPL 3). The sleep stage of the user can be detected by focusing on the fact that the appearance distribution and the transition probability of sleep stages change depending on the elapsed time from bedtime.

Also, if the sensor 105 is a 3-axis acceleration sensor, the sleep stage detection unit no uses machine learning or the like to classify the sleep stage from the user's activity count and idleness periods based on the 3-axis acceleration data acquired by the sensor data acquisition unit 10 (see NPL 2). For example, the sleep stage detection unit no can integrate and use the Cole algorithm, which is a technique for determining the sleep stage based on the activity count, and the ESS algorithm that is based on the idleness period (see NPL 2).

Also, if the sensor 105 is a microwave sensor, the sleep stage detection unit no can detect the sleep stage by calculating the fluctuation of the user's activity and breathing interval based on microwaves acquired by the sensor data acquisition unit 10 (see NPL 4 and 5). Specifically, a microwave radar is used to detect minute vibrations associated with respiration and heartbeat activity appearing at the user's body surface, and the sleep stage is detected from set parameters.

Also, if the sensor 105 is a pressure sensor, the sleep stage detection unit no can detect pressure changes caused by the user's heartbeat vibration, respiratory movement, snoring vibration, and body movement based on pressure data acquired by the sensor data acquisition unit 10, and classify the sleep stage based on such data (see NPL 6).

The sleep stage detection unit no detects three stages, namely awake, light sleep, and deep sleep, for example. For example, the sleep stage detection unit no can detect wake (stage W), REM sleep (stage V), and non-REM sleep (stages I-IV) stages based on the Rechtschaffen & Kales sleep stage classification (see NPL 4).

More specifically, the sleep stage detection unit no may be configured to detect that the user has fallen asleep when the value of the sensor data corresponding to the stage I included in non-REM sleep is detected. The sleep stage detection unit no records the sleep onset time in the storage unit 12 if the user's sleep onset has been detected.

Also, the sleep stage detection unit no detects that the user is in the deep sleep stage if the detected value of the sensor data corresponds to the stages III or IV during non-REM sleep. Note that the reference for detecting that the user is in the deep sleep stage may include a sleep depth that corresponds to stage II in non-REM sleep. The references for detecting the sleep stage of the user are not limited to the above description.

The first determination unit 111 determines whether or not it is time to change the position of the user based on the sleep stage of the user detected by the sleep stage detection unit no. More specifically, the first determination unit 111 determines that it is time to change the position if the user is in the deep sleep stage.

If the position of the user is changed when the user is in a deeper sleep stage, the user is not likely to wake up, and therefore embodiments of the present invention can suppress an interruption of the user's sleep. Also, it has been reported that if the position of the user is changed while the user is in the deep sleep stage, then even if the user awakens, the user can fall asleep again in a shorter time than in the case where the position is changed in other sleep stages (see NPL 7).

The storage unit 12 stores sensor data that indicates biological information of the user acquired by the sensor data acquisition unit 10. The storage unit 12 also stores the sleep stage of the user detected by the sleep stage detection unit no together with time information.

If the first determination unit 111 determined that it is time to change the position of the user, the notification unit 13 gives a notification of that fact. For example, the notification unit 13 can display a text message indicating that it is time to change the position of the user on a display device 109, or give a notification by blinking an LED or outputting voice from a speaker (not shown), for example. Note that the method of realizing the notification unit 13 is not limited to these examples, as long as the notification is given in a manner recognizable by the caregiver who cares for the user.

The transmission/reception unit 14 receives sensor data that indicates biological information of the user and was measured by the later-described sensor 105. The transmission/reception unit 14 can also send, to the outside via a communication network, a notification generated by the notification unit 13 indicating that it is time to change the position of the user.

### Computer Configuration of Long-Term Care Support Device

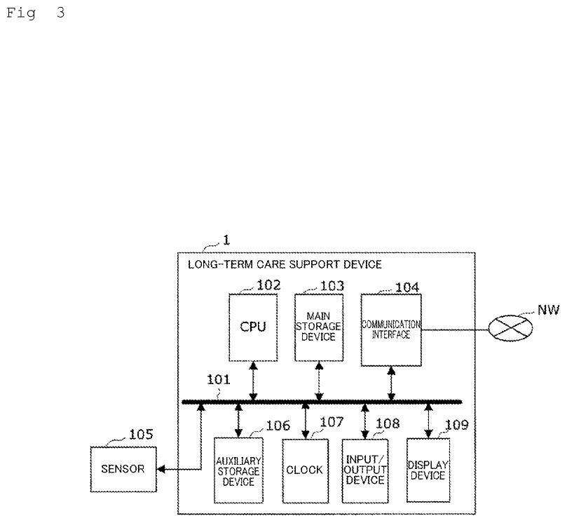

Next, a computer configuration for realizing the long-term care support device 1 having the above-described functions will be described with reference to FIG. 3.

As shown in FIG. 2, the long-term care support device 1 can be realized by a computer including, for example, a CPU 102, a main storage device 103, a communication interface 104, an auxiliary storage device 106, a clock 107, and an input/output device 108, which are connected via a bus 101, as well as a program that controls these hardware resources. In the long-term care support device 1, an externally-provided sensor 105 and a display device 109 provided inside the long-term care support device 1 are connected to each other via the bus 101.

A program for causing the CPU 102 to perform various types of control and operations is stored in the main storage device 103 in advance. Functions of the long-term care support device 1 including the data analysis unit 11 shown in FIG. 1 are realized by the CPU 102 and the main storage device 103.

The communication interface 104 is an interface circuit for performing communication with various external electronic devices via a communication network NW.

A calculation interface and an antenna that correspond to a wireless data communication standard such as LTE, 3G, wireless LAN, or Bluetooth (registered trademark) are used As the communication interface 104, for example. The transmission/reception unit 14 described with reference to FIG. 1 is realized by the communication interface 104.

The sensor 105 is realized by, for example, a heart rate monitor, an electrocardiograph, a 3-axis acceleration sensor, a microwave sensor, a pressure sensor, or a combination thereof.

The auxiliary storage device 106 is constituted by a readable and writable storage medium and a drive device for reading and writing various types of information such as programs and data to and from the storage medium. A semiconductor memory such as a hard disk or a flash memory can be used as the storage medium in the auxiliary storage device 106.

The auxiliary storage device 106 has a storage area for storing biological information measured by the sensor 105, and a program storage area for storing a program for causing the long-term care support device 1 to perform biological information analysis processing. The storage unit 12 described with reference to FIG. 1 is realized by the auxiliary storage device 106. Further, a backup area for backing up the above-described data, programs, and the like may be provided.

The clock 107 is constituted by a built-in clock that is built in the computer or the like, and measures the time. Time information obtained by the clock 107 is recorded in association with a detected sleep stage of the user. Time information obtained by the clock 107 is also used for sampling biological information and the like.

The input/output device 108 is constituted by an I/O terminal that receives signals from external devices such as the sensor 105 and the display device 109, and outputs signals to the external devices.

The display device 109 is realized by a liquid crystal display or the like. Also, the display device 109 can display a notification given by the notification unit 13 described with reference to FIG. 1 on the screen.

### Long-Term Care Support Method

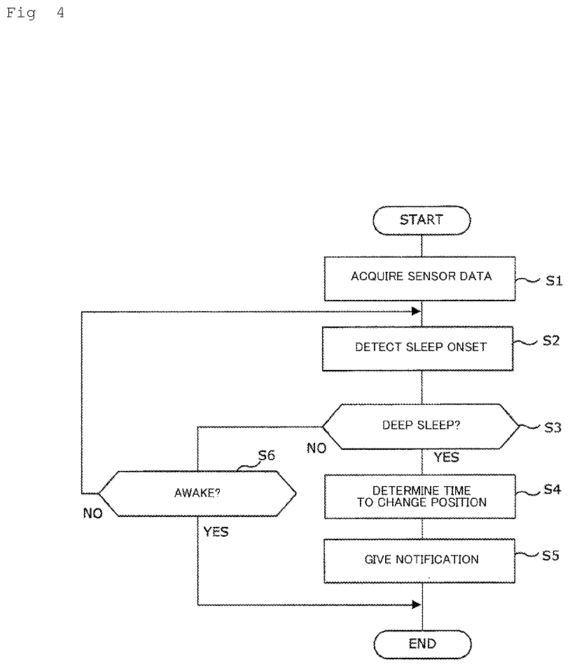

Next, operations of the long-term care support device 1 having the above-described configuration will be described with reference to the flowchart of FIG. 4. First, for example, the user goes to bed with the sensor 105 attached, and the following processing is executed.

The sensor data acquisition unit 10 acquires biological information of the user measured by the sensor 105 via the transmission/reception unit 14 (step S1). The acquired biological information is stored in the storage unit 12. Note that the sensor data acquisition unit 10 can remove noise from the acquired biological information and can also perform processing for converting the biological information from an analog signal into a digital signal.

Next, the sleep stage detection unit no detects that the user has fallen asleep according to a preset reference based on the biological information of the user acquired by the sensor data acquisition unit 10 (step S2). Specifically, the sleep stage detection unit no detects that the biological information of the user indicates the awake stage, the light sleep stage, or the deep sleep stage. For example, if the sleep stage corresponds to stage I of non-REM sleep based on the above-mentioned Rechtschaffen & Kales sleep stage classification, the sleep stage detection unit no may detect that the user is in the sleep onset stage. If sleep onset is detected by the sleep stage detection unit no, the sleep onset is recorded in the storage unit 12 together with the time information.

Thereafter, if the sleep stage detection unit no detects that the user is in the deep sleep stage (step S3: YES), the first determination unit 111 determines that it is time to change the position of the user (step S4). Next, the notification unit 13 gives a notification that it is time to change the position of the user based on the determination made by the first determination unit 111 (step S5). Accordingly, it is possible to prompt the caregiver or the like to change the position of the user at a more suitable timing for changing the position of the user.

On the other hand, in the case where the deep sleep stage is not detected based on the biological information of the user (step S3: NO), if the sleep stage detection unit no then detects that the user is in the awake stage (step S6: YES), the processing ends. On the other hand, if the awake stage is not detected in step S6 (step S6: NO), the processing moves to step S2.

### Long-Term Care Support System

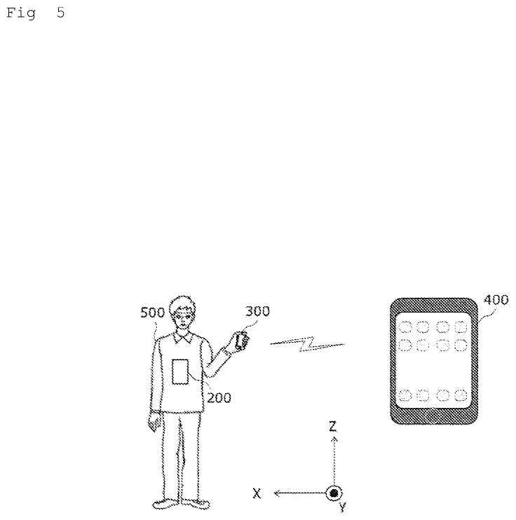

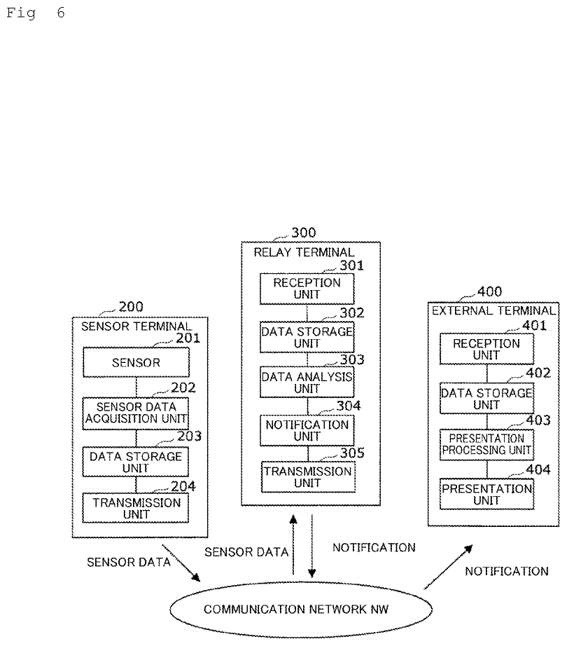

Next, a long-term care support system that specifically includes the long-term care support device 1 according to embodiments of the present invention will be described with reference to FIGS. 5 and 6.

As shown in FIG. 5, the long-term care support system includes, for example, a sensor terminal 200 attached to a user 500, a relay terminal 300, and an external terminal 400. All or any of the sensor terminal 200, the relay terminal 300, and the external terminal 400 are provided with functions of units of the long-term care support device 1 such as the data analysis unit 11 described with reference to FIGS. 1 and 2. Note that in the following, it is assumed that the relay terminal 300 includes the data analysis unit ii and the notification unit 13 described with reference to FIG. 1.

### Function Blocks of Sensor Terminal

The sensor terminal 200 includes a sensor 201, a sensor data acquisition unit 202, a data storage unit 203, and a transmission unit 204. The sensor terminal 200 is arranged on the trunk of the body of the user 500, for example, and measures biological information. The sensor terminal 200 transmits the measured biological information of the user 500 to the relay terminal 300 via the communication network NW.

The sensor 201 is realized by a heart rate monitor, a 3-axis acceleration sensor, or the like. As shown in FIG. 4, the three axes of the acceleration sensor included in the sensor 201 includes an X axis parallel with the left-right direction of the body, a Y axis parallel with the front-rear direction of the body, and a Z axis parallel with the up-down direction of the body. The sensor 201 corresponds to the sensor 105 described with reference to FIG. 1.

The sensor data acquisition unit 202 acquires the biological information measured by the sensor 201. More specifically, the sensor data acquisition unit 202 removes noise from the acquired biological information and performs sampling processing to obtain time-series data in the biological information, which is a digital signal. The sensor data acquisition unit 202 corresponds to the sensor data acquisition unit 10 described with reference to FIG. 1.

The data storage unit 203 stores the biological information measured by the sensor 201 and the time-series data of the biological information, which is a digital signal, that was processed by the sensor data acquisition unit 202. The data storage unit 203 corresponds to the storage unit 12 (FIG. 1).

The transmission unit 204 transmits the biological information stored in the data storage unit 203 to the relay terminal 300 via the communication network NW. The transmission unit 204 includes, for example, a communication circuit for performing wireless communication compliant with a wireless data communication standard such as LTE, 3G, wireless LAN (Local Area Network), or Bluetooth (registered trademark). The transmission unit 204 corresponds to the transmission/reception unit 14 (FIG. 1).

### Function Blocks of Relay Terminal

The relay terminal 300 includes a reception unit 301, a data storage unit 302, a data analysis unit 303, a notification unit 304, and a transmission unit 305. The relay terminal 300 analyzes the biological information of the user 500 received from the sensor terminal 200. Furthermore, the relay terminal 300 detects the sleep stage of the user 500 based on the biological information of the user 500. Also, the relay terminal 300 gives a notification that it is time to change the position of the user 500 if the user 500 is in the deep sleep stage based on the detected sleep stage of the user 500. The generated notification is transmitted to the external terminal 400.

The relay terminal 300 is realized by a smartphone, a tablet, a laptop computer, or the like.

The reception unit 301 receives biological information from the sensor terminal 200 via the communication network NW. The reception unit 301 corresponds to the transmission/reception unit 14 (FIG. 1).

The data storage unit 302 stores the biological information of the user 500 received by the reception unit 301 and the sleep stage information of the user 500 detected by the data analysis unit 303. The data storage unit 302 corresponds to the storage unit 12 (FIG. 1).

The data analysis unit 303 analyzes the biological information of the user 500 received by the reception unit 301, and detects the sleep stage of the user 500 based on a preset reference. Also, if it was detected that the user 500 is in the deep sleep stage, the data analysis unit 303 determines that it is time to change the position of the user 500. The data analysis unit 303 corresponds to the data analysis unit ii that includes the sleep stage detection unit no and the first determination unit 111 described with reference to FIGS. 1 and 2.

If the data analysis unit 303 detected the deep sleep stage of the user 500 and determined that it is time to change the position of the user 500, the notification unit 304 gives a notification of that fact. The notification unit 304 corresponds to the notification unit 13 described with reference to FIG. 1.

The transmission unit 305 transmits the notification generated by the notification unit 304 to the external terminal 400 via the communication network NW. The transmission unit 305 corresponds to the transmission/reception unit 14 (FIG. 1).

### Function Blocks of External Terminal

The external terminal 400 includes a reception unit 401, a data storage unit 402, a presentation processing unit 403, and a presentation unit 404. The external terminal 400 presents a notification indicating that it is time to change the position of the user 500, which was received from the relay terminal 300 via the communication network NW. The external terminal 400 is a terminal carried by the caregiver.

Similarly to the relay terminal 300, the external terminal 400 is realized by a smartphone, a tablet, a laptop computer, or the like. The external terminal 400 includes a display device that displays the received notification. Note that besides the display device, notification information may be presented by a voice output device, a light source, or the like.

The reception unit 401 receives a notification from the relay terminal 300 via the communication network NW. The reception unit 401 corresponds to the transmission/reception unit 14 (FIG. 1).

The data storage unit 402 stores the notification received by the reception unit 401. The data storage unit 402 corresponds to the storage unit 12 (FIG. 1).

The presentation processing unit 403 controls the display format of the notification indicating that it is time to change the position of the user 500 by using a style sheet or the like. The presentation processing unit 403 corresponds to the notification unit 13 described with reference to FIG. 1.

The presentation unit 404 outputs a notification indicating that it is time to change the position of the user 500 based on the instruction from the presentation processing unit 403. The presentation unit 404 may display text information on a display device, or output a notification by an alert sound or the like from a speaker (not shown) provided in the external terminal 400. Alternatively, the presentation unit 404 can present the notification by a method that the caregiver can recognize, such as vibration or light. The presentation unit 404 corresponds to the notification unit 13 described with reference to FIG. 1.

In this way, the long-term care support system according to embodiments of the present invention has a configuration in which functions of the long-term care support device 1 are distributed to the sensor terminal 200, the relay terminal 300, and the external terminal 400, and various types of processing are also performed in a distributed manner, including processing from the acquisition of biological information of the user 500 to the detection of the sleep stage, the determination of the time to change the position of the user 500, the generation of a notification, and the presentation of the notification.

### Operation Sequency of Long-Term Care Support System

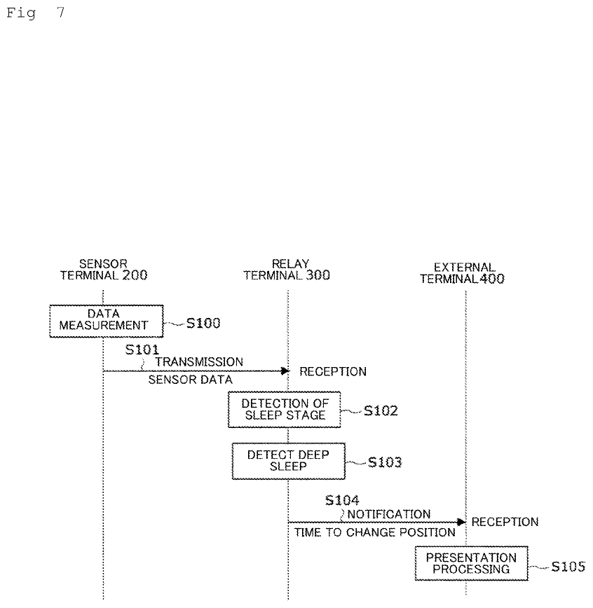

Next, operations of the long-term care support system having the above-described configuration will be described with reference to the sequence diagram of FIG. 7.

As shown in FIG. 7, first, the sensor terminal 200 is attached to the user 500, for example, and measures biological information such as heart rate variability and triaxial acceleration (step S100). The sensor terminal 200 obtains a digital signal from the measured biological information, and removes noise as necessary.

Next, the sensor terminal 200 transmits the biological information to the relay terminal 300 via the communication network NW (step S101). When the relay terminal 300 receives the biological information from the sensor terminal 200, the relay terminal 300 detects the sleep stage of the user 500 based on the biological information and a preset reference (step S102). More specifically, the data analysis unit 303 of the relay terminal 300 detects that the user 500 is in the sleep onset stage and records the time information.

Next, upon determining that the user 500 is in the deep sleep stage in step S102, the data analysis unit 303 determines that it is time to change the position of the user 500 (step S103). Thereafter, the relay terminal 300 transmits, to the external terminal 400, a notification that it is time to change the position of the user 500 via the communication network NW (step S104). Upon receiving the notification information, the external terminal 400 executes presentation processing (step S105). The external terminal 400 causes the display device 109 to display text information indicating that it is time to change the position of the user 500, for example.

As described above, the long-term care support device 1 according to the first embodiment detects the user's sleep stage based on biological information of the user measured by the sensor 105, and upon detecting that the user is in the deep sleep, determines that it is time to change the position of the user, and gives a notification. Because the caregiver can be notified that the position of the user is to be changed when the user is in the deep sleep stage, it is possible to change the posture of the sleeping user while suppressing interruption of the user's sleep.

### Second Embodiment

Next, a second embodiment of the present invention will be described. Note that in the following description, configurations that are the same as those in the first embodiment described above are designated by the same reference numerals, and the description thereof will be omitted.

In the first embodiment, it is determined that it is time to change the position of the user if it is detected that the user is in the deep sleep stage. In contrast, in the second embodiment, the elapse of a preset period (first period) is measured from the time when the user falls asleep, and even if it is not detected that the user is in the deep sleep stage within that period, it is determined that it is time to change the position of the user, and a notification is given.

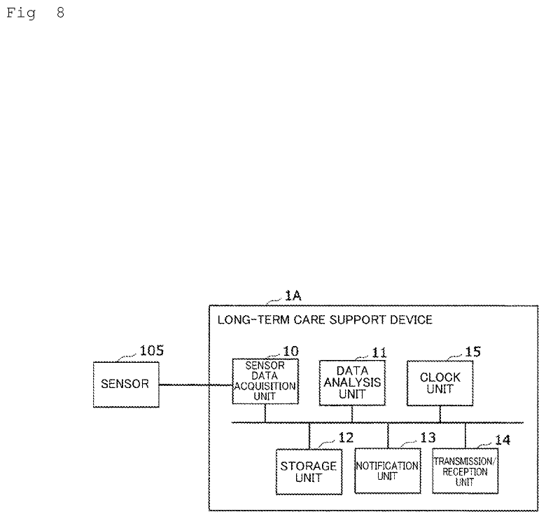

As shown in FIG. 8, the configuration of a long-term care support device 1A according to the second embodiment is different from that of the first embodiment in that it further includes a clock unit 15. The following description focuses on configurations different from those of the first embodiment.

The clock unit 15 measures the elapse of a preset period from the time when the sleep stage detection unit no detects that the user has fallen asleep. For example, the clock unit 15 can measure four hours from the time when the user falls asleep. Note that the sleep stage detection unit no can set the length of the period to two hours, three hours, or the like, as long as it is possible to find out a suitable time for performing a position change for caring for the user's bedsores.

Also, as described in NPL 8, it is generally said that REM sleep and non-REM sleep are repeated in a 90-minute cycle, for example. By using three hours as the preset period, the user is more likely to be in a deeper sleep stage, it is possible to set a more suitable period for preventing bedsores.

If the time measured by the clock unit 15 has reached the set period from the sleep onset time, the first determination unit 111 determines that it is time to change the position of the user.

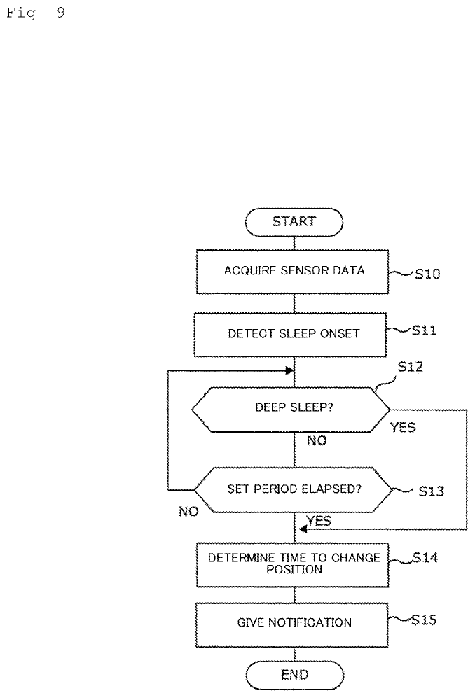

Next, operations of the long-term care support device 1A of the present embodiment having the above-described configuration will be described with reference to the flowchart of FIG. 9.

First, a sensor 105 that includes a heart rate monitor, a 3-axis acceleration sensor, or the like is attached to the user, and the following processing is executed while the user is in bed.

The sensor data acquisition unit 10 acquires biological information of the user measured by the sensor 105 via the transmission/reception unit 14 (step S10). The acquired biological information is stored in the storage unit 12. Note that the sensor data acquisition unit 10 can remove noise from the acquired biological information and can also perform processing for converting the biological information from an analog signal into a digital signal.

Next, the sleep stage detection unit 110 detects that the user has fallen asleep according to a preset reference based on the biological information of the user acquired by the sensor data acquisition unit 10 (step S11). If sleep onset is detected by the sleep stage detection unit 110, the sleep onset is recorded in the storage unit 12 together with the time information. Also, the clock unit 15 measures the elapse of a preset period from the sleep onset time.

Thereafter, if the sleep stage detection unit 110 has not detected that user is in the deep sleep stage (step S12: NO), and the set period elapses (step S13: YES), the first determination unit 111 determines that it is time to change the position of the user (step S14). Next, the notification unit 13 gives a notification that it is time to change the position of the user based on the determination made by the first determination unit 111 (step S15). Accordingly, the caregiver can change the position of the user at a more suitable time for changing the position of the user.

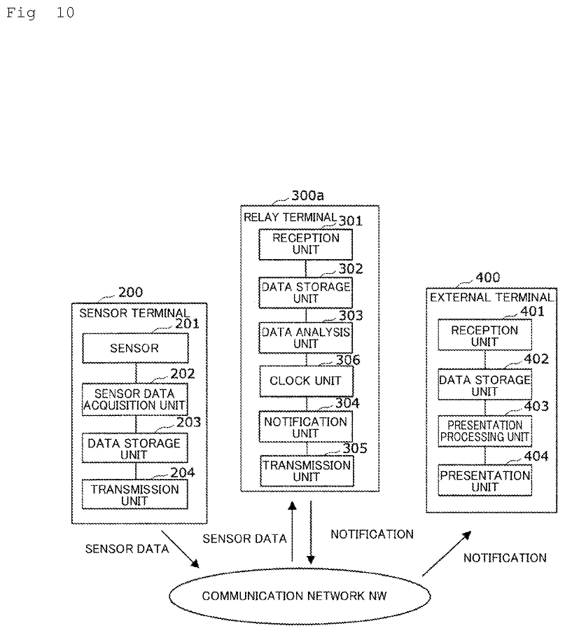

Next, a long-term care support system that specifically includes the long-term care support device 1A according to the present embodiment will be described with reference to FIG. 10. The configurations of the sensor terminal 200 and the external terminal 400 provided in the long-term care support system are similar to those in the first embodiment.

A relay terminal 300a according to the present embodiment is different in configuration from the relay terminal 300 according to the first embodiment in that it further includes a clock unit 306. The clock unit 306 measures the elapse of a set period such as four hours from the time when the data analysis unit 303 (sleep stage detection unit no) detects that the user 500 has fallen asleep.

As described above, according to the second embodiment, after the set period has elapsed since when the user fell asleep, even if the user has not reached deep sleep and the deep sleep stage has not been detected, it is determined to be time to change the position of the user, and a notification is given. Accordingly, it is possible to more reliably give a notification of the time to change the position of the user.

### Third Embodiment

Next, a third embodiment of the present invention will be described. Note that in the following description, configurations that are the same as those in the first and second embodiments described above are designated by the same reference numerals, and the description thereof will be omitted.

In the second embodiment, it is described that it is determined that it is time to change the position of the user if the preset period has elapsed since when the user fell asleep. However, in the third embodiment, a second determination unit (a change determination unit) 112 is provided to determine whether or not the position of the user changed. If it has been determined that the position of the user changed, the first determination unit 111 makes a determination regarding whether or not it is time to change the position of the user again.

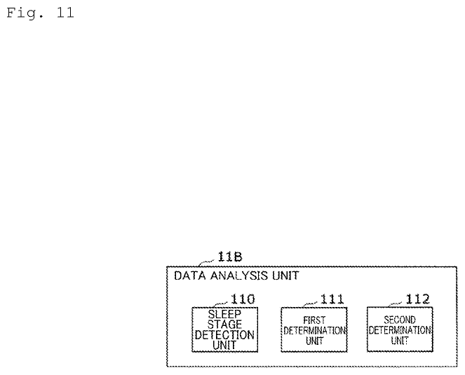

As shown in FIG. 11, a data analysis unit 11B according to the present embodiment includes the sleep stage detection unit 110, the first determination unit 111, and a second determination unit 112. Other configurations of the long-term care support device 1B are similar to those of the long-term care support device 1A according to the second embodiment.

The second determination unit 112 determines whether or not the position of the user has changed. More specifically, if the sleep stage detection unit no detects that the user woke up, the second determination unit 112 can determine that the position of the user has changed. Alternatively, the second determination unit 112 may determine that the position of the user has changed if the notification unit 13 gave a notification that it is time to change the position of the user, and furthermore the sleep stage detection unit no detected that the user woke up.

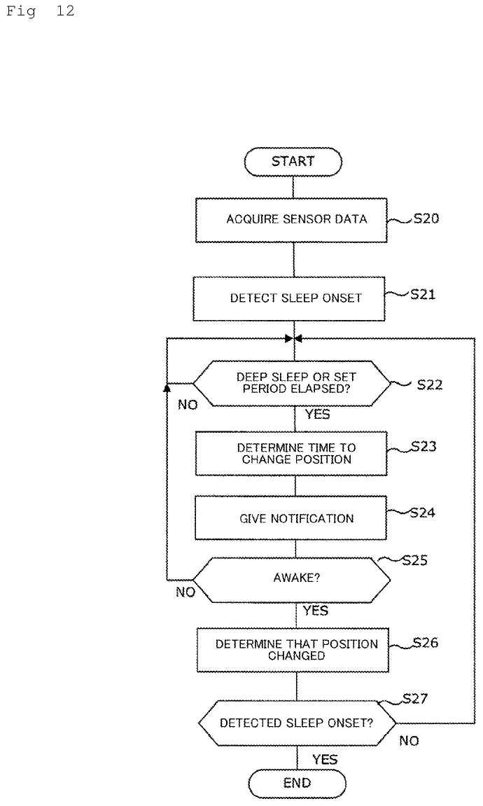

Next, operations of the long-term care support device 1A that includes the second determination unit 112 will be described with reference to the flowchart of FIG. 12. First, the sensor 105 that includes a heart rate monitor, a 3-axis acceleration sensor, or the like is attached to the user, and the following processing is executed while the user is in bed.

The sensor data acquisition unit 10 acquires biological information of the user measured by the sensor 105 via the transmission/reception unit 14 (step S20). The acquired biological information is stored in the storage unit 12. Note that the sensor data acquisition unit 10 can remove noise from the acquired biological information and can also perform processing for converting the biological information from an analog signal into a digital signal.

Next, the sleep stage detection unit no detects that the user has fallen asleep according to a preset reference based on the biological information of the user acquired by the sensor data acquisition unit 10 (step S21). If sleep onset is detected by the sleep stage detection unit no, the sleep onset is recorded in the storage unit 12 together with the time information. Also, the clock unit 15 measures the elapse of a preset period from the sleep onset time.

Thereafter, if the sleep stage detection unit no detects that the user is in the deep sleep stage, or the preset period has elapsed since the sleep onset time (step S22: YES), the first determination unit 111 determines that it is time to change the position of the user (step S23). Next, the notification unit 13 gives a notification that it is time to change the position of the user based on the determination made by the first determination unit 111 (step S24).

Thereafter, if the sleep stage detection unit no has detected that the user woke up (step S25: YES), the second determination unit 112 determines that the position of the user changed (step S26). Then, after the elapse of a certain period of time, if the sleep stage detection unit no has detected that the user fell asleep again (step S27: YES), the processing moves to step S22. Then, in step S22, if it has been detected that the user is in the deep sleep stage, or the time measured by the clock unit (second clock unit) 15 has reached a set period (second period), the first determination unit 111 again determines that it is time to change the position of the user (step S23).

Note that it is sufficient that the clock unit 15 measures the elapse of a set period, such as two hours, three hours, or four hours.

As described above, according to the third embodiment, it is determined whether or not the position of the user has changed, and then, after it was detected that the user fell asleep, if the set period has elapsed or it was detected that the user is in the deep sleep stage, it is determined that it is time to change the position of the user, and a notification is given. Accordingly, even if the user sleeps for a long time, it is possible to give a notification of the time to change the position of the user at a more appropriate interval while also suppressing interruption of the user's sleep.

### Fourth Embodiment

Next, a fourth embodiment of the present invention will be described. Note that in the following description, configurations that are the same as those in the first to third embodiments described above are designated by the same reference numerals, and the description thereof will be omitted.

In the first embodiment, it is described that if it was detected that the user is in the deep sleep stage, it is determined that it is time to change the position of the user, and a notification is given. In contrast, in the fourth embodiment, if it is detected that the user has awakened after the user has fallen asleep, it is determined that it is time to change the position of the user, and the notification is given.

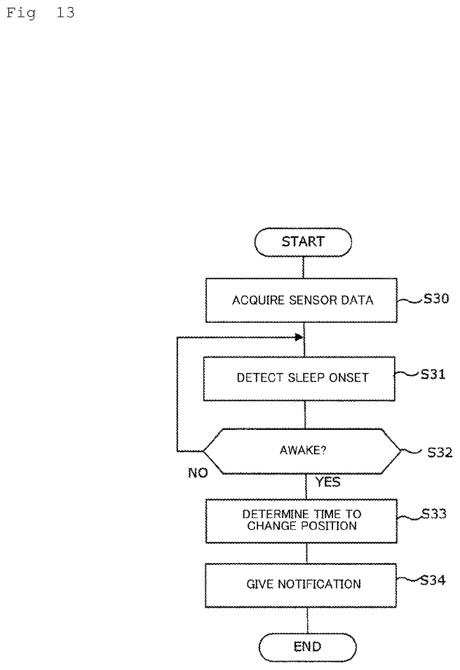

The configuration of the long-term care support device 1 according to the fourth embodiment is similar to that of the first embodiment. Hereinafter, operations of the long-term care support device 1 of the present embodiment will be described with reference to the flowchart of FIG. 13.

First, the sensor 105 that includes a heart rate monitor, a 3-axis acceleration sensor, or the like is attached to the user, and the following processing is executed while the user is in bed.

The sensor data acquisition unit 10 acquires biological information of the user measured by the sensor 105 via the transmission/reception unit 14 (step S30). The acquired biological information is stored in the storage unit 12. Note that the sensor data acquisition unit 10 can remove noise from the acquired biological information and can also perform processing for converting the biological information from an analog signal into a digital signal.

Next, the sleep stage detection unit no detects that the user has fallen asleep according to a preset reference based on the biological information of the user acquired by the sensor data acquisition unit 10 (step S31). If sleep onset is detected by the sleep stage detection unit no, the sleep onset is recorded in the storage unit 12 together with the time information. Also, the clock unit 15 measures the elapse of a preset period from the sleep onset time.

Thereafter, if the sleep stage detection unit no detects that the user woke up (step S32: YES), the first determination unit 111 determines that it is time to change the position of the user (step S33). Next, the notification unit 13 gives a notification that it is time to change the position of the user based on the determination made by the first determination unit 111 (step S34).

As described above, according to the fourth embodiment, after the user has fallen asleep, if it is detected that the user woke up, it is rational to change the posture of the user before the user falls asleep again, and therefore it is possible to notify the caregiver to change the position of the user at a more appropriate timing. Accordingly, the caregiver can change the position of the user before the user falls asleep again.

### Fifth Embodiment

Next, a fifth embodiment of the present invention will be described. Note that in the following description, configurations that are the same as those in the first to fourth embodiments described above are designated by the same reference numerals, and the description thereof will be omitted.

In the first to fourth embodiments, the case of determining the time to changing the position of the user and then giving a notification is described. In contrast, in the fifth embodiment, the long-term care support device 1B further includes a position change processing unit 16 that outputs a control signal to a movable device, which is provided externally and is for changing the position of the user, based on a determination result. The following description focuses on configurations different from those of the first to fourth embodiments.

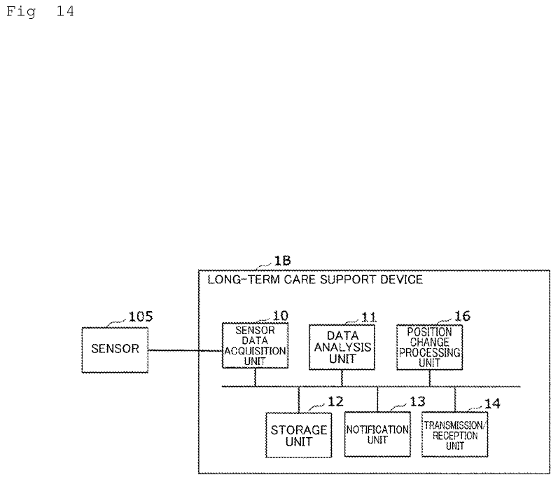

As shown in FIG. 14, the long-term care support device 1B according to the present embodiment further includes the position change processing unit 16. Other configurations of the long-term care support device 1B are similar to those in the first embodiment.

The position change processing unit 16 outputs a control signal to a movable device for changing the position of the user based on a notification indicating that it is time to change the position of the user. The movable device is a device capable of changing the position of the user using electric power, such as a movable bed.

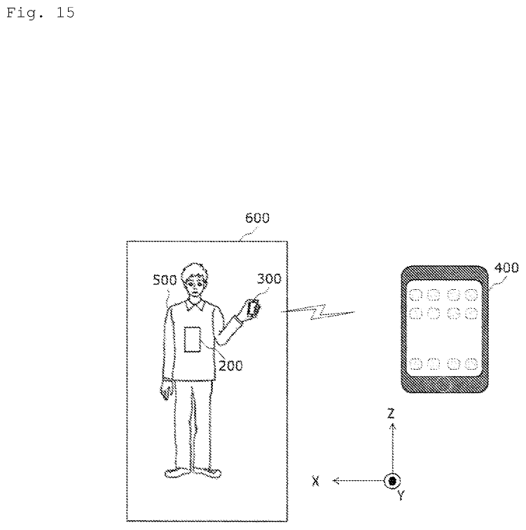

FIG. 15 shows an overview of a long-term care support system that specifically includes the long-term care support device 1B according to the present embodiment. The long-term care support system includes the sensor terminal 200 that measures the heart rate, triaxial acceleration, or the like of the user 500, the relay terminal 300, the external terminal 400, and a movable bed 600.

For example, a bed having an inclining floor as shown in NPL 9 can be used as the movable bed 600.

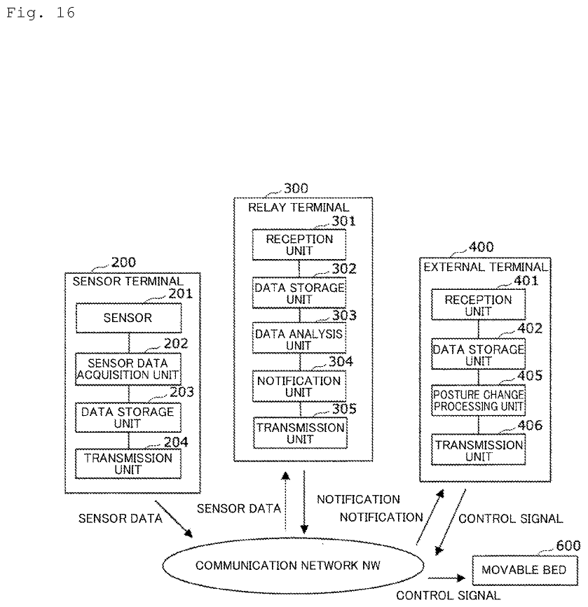

As shown in FIG. 16, the sensor terminal 200, the relay terminal 300, the external terminal 400, and the movable bed 600 are connected to each other via the communication network NW. Note that the configurations of the sensor terminal 200 and the relay terminal 300 are similar to those of the first embodiment. In the present embodiment, the external terminal 400 includes a position change processing unit 405 and a transmission unit 406.

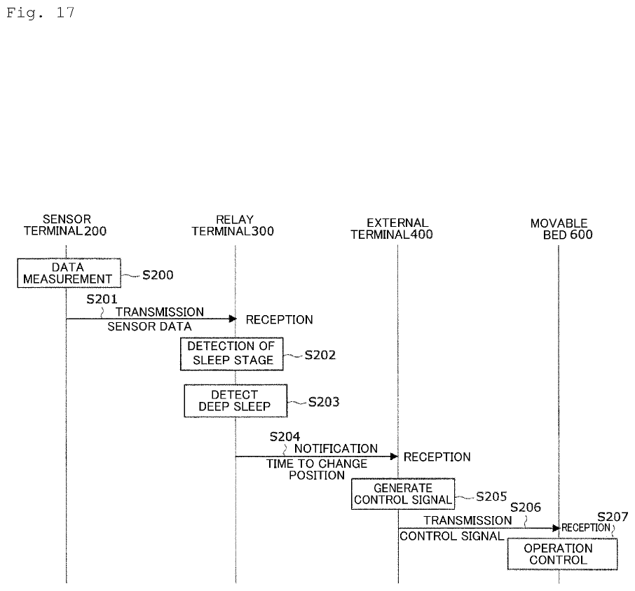

FIG. 17 shows a sequence that illustrates operations of the long-term care support system having the above-described configuration.

As shown in FIG. 17, first, the sensor terminal 200 is attached to the user 500, for example, and measures biological information such as heart rate variability and triaxial acceleration (step S200). The sensor terminal 200 obtains a digital signal from the measured biological information, and removes noise as necessary.

Next, the sensor terminal 200 transmits the biological information to the relay terminal 300 via the communication network NW (step S101). When the relay terminal 300 receives the biological information from the sensor terminal 200, the relay terminal 300 detects the sleep stage of the user 500 based on the biological information and a preset reference (step S202). More specifically, the data analysis unit 303 of the relay terminal 300 detects that the user 500 is in the sleep onset stage and records the time information.

Next, upon detecting that the user 500 is in the deep sleep stage, the data analysis unit 303 determines that it is time to change the position of the user 500 (step S203). Thereafter, the relay terminal 300 transmits, to the external terminal 400, a notification that it is time to change the position of the user 500 via the communication network NW (step S204). Upon receiving the notification information, the external terminal 400 generates a control signal for the movable bed 600 (step S205).

Next, the external terminal 400 transmits the control signal to the movable bed 600 using the transmission unit 406 (step S206). Thereafter, the movable bed 600 changes the position of the user by tilting the floor of the bed based on the received control signal (step S207).

As described above, according to the fifth embodiment, the present invention transmits a control signal to a movable device such as the movable bed 600 based on the determination that it is time to change the position of the user, thus causing the movable device to change the position of the user. For this reason, according to the fifth embodiment of the present invention, it is possible to realize user position changing while reducing the burden on the sleeping user and also reducing the burden on the caregiver.

Although embodiments of a long-term care support device, a long-term care support method, and a long-term care support system of the present invention have been described above, the present invention is not limited to the described embodiments, and various modifications that can be arrived at by those skilled in the art can be made within the scope of the invention described in the claims.

It should be noted that the described embodiments can be realized in combination with each other. Also, in the long-term care support system given as a specific example of realizing the long-term care support device 1, the case was described in which the relay terminal 300 includes the sleep stage detection unit no and the first determination unit 111. However, the sleep stage detection unit no and the first determination unit 111 may be provided in either the sensor terminal 200 or the external terminal 400.

Also, functions of the data analysis unit 11 (e.g., the sleep stage detection unit no and first determination unit 111) may be distributed among the sensor terminal 200, the relay terminal 300, and the external terminal 400.

### REFERENCE SIGNS LIST

1 Long-term care support device
10, 202 Sensor data acquisition unit
11, 303 Data analysis unit
12 Storage unit
13, 304 Notification unit
14 Transmission/reception unit
110 Sleep stage detection unit
111 First determination unit
101 Bus
102 CPU
103 Main storage device
104 Communication interface
105, 201 Sensor
106 Auxiliary storage device
107 Clock
108 Input/output device
109 Display device
200 Sensor terminal
300 Relay terminal
400 External terminal
203, 302, 402 Data storage unit
204, 305 Transmission unit
301, 401 Reception unit
403 Presentation processing unit.

1.-8. (canceled)

9. A long-term care support device comprising:
a sensor data acquisition device configured to acquire biological information of a user that was measured by a sensor;
a sleep stage detector configured to detect a sleep stage of the user based on the biological information;
a timing determination device configured to determine that it is time to change a position of the user in response to the sleep stage of the user being a deep sleep stage; and
a notification device configured to, in response to the timing determination device determined that it is time to change the position of the user, give a notification that it is time to change the position of the user.

10. The long-term care support device according to claim 9, wherein:
the sleep stage detector is further configured to detect that the user is in a sleep onset stage;
the long-term care support device further comprises a first clock configured to:
measure a preset first period from a first time when it was detected that the user is in the sleep onset stage; and
in response to a first elapsed time measured by the first clock has reached the preset first period, the timing determination device is configured to determine that it is time to change the position of the user.

11. The long-term care support device according to claim 9, further comprising:
a change determination device configured to determine that the position of the user changed based on the sleep stage of the user; and
a second clock configured to measure a preset second period from a second time when the change determination device detected that the position of the user changed, wherein in response to the sleep stage of the user is the deep sleep stage or in response to a second elapsed time measured by the second clock has reached the preset second period, the timing determination device is configured to determine that it is time to change the position of the user.

12. The long-term care support device according to claim 9, wherein in response to the sleep stage detector detecting that the user is in a sleep onset stage and thereafter detects that the user is in an awake stage, the timing determination device is configured to determine that it is time to change the position of the user.

13. The long-term care support device according to claim 9, further comprising:
a position change processing device configured to output a control signal to a movable device configured to change the position of the user, based on the notification that it is time to change the position of the user.

14. The long-term care support device according to any one of claim 9, wherein the sensor is a heart rate monitor, a 3-axis acceleration sensor, a microwave sensor, a pressure sensor, or a combination thereof.

15. A long-term care support method comprising:
acquiring biological information of a user that was measured by a sensor;
detecting a sleep stage of the user according to a preset reference based on the biological information;
determining that it is time to change a position of the user in response to detecting the sleep stage of the user is a deep sleep stage; and
in response to determining that it is time to change the position of the user, giving a notification that it is time to change the position of the user.

16. The long-term care support method according to claim 15, further comprising:
detecting that the user is in a sleep onset stage;
measuring, by a first clock, a preset first period from a first time when it was detected that the user is in the sleep onset stage; and
in response to a first elapsed time measured by the first clock has reached the preset first period, determining that it is time to change the position of the user.

17. The long-term care support method according to claim 15, further comprising:
determining that the position of the user changed; and
measuring, by a second clock, a preset second period from a second time of determining that the position of the user changed, wherein in response to the sleep stage of the user is the deep sleep stage or in response to a second elapsed time measured by a second clock has reached the preset second period, determining that it is time to change the position of the user.

18. The long-term care support method according to claim 15, wherein in response to detecting that the user is in a sleep onset stage and thereafter detects that the user is in an awake stage, determining that it is time to change the position of the user.

19. The long-term care support method according to claim 15, wherein the sensor is a heart rate monitor, a 3-axis acceleration sensor, a microwave sensor, a pressure sensor, or a combination thereof.

20. A long-term care support system comprising:
a sensor terminal configured to output, to an external device, biological information of a user that was measured by a sensor;
a relay terminal configured to receive the biological information output by the sensor terminal, and output the biological information to an external device; and
an external terminal configured to receive the biological information that was output by the sensor terminal or the relay terminal, and display the biological information on a display device, wherein at least any one of the sensor terminal, the relay terminal, and the external terminal includes:
a sensor data acquisition device configured to acquire the biological information;
a sleep stage detector configured to detect a sleep stage of the user according to a preset reference based on the biological information;
a timing determination device configured to determine that it is time to change a position of the user in response to the sleep stage of the user being a deep sleep state; and
a notification device configured to in response to the timing determination device determining that it is time to change the position of the user, give a notification that it is time to change the position of the user.
